(19) United States
(12) Patent Application Publication
(10) Pub. No.: US 2015/0202383 A1
(43) Pub. Date: Jul. 23, 2015

(54) OFF-AXIS OPTICAL SENSOR FOR DETECTING INFUSION PUMP CASSETTE

(71) Applicant: Zevex, Inc., Salt Lake City, UT (US)

(72) Inventors: Jeffery T. Juretich, Herriman, UT (US); Michael Marshall, Herriman, UT (US); Michael Elwood, Farmington, UT (US); Daniel A. Martel, Stansbury, UT (US); Jeffrey Geisler, Salt Lake City, UT (US)

(21) Appl. No.: 14/161,830

(22) Filed: Jan. 23, 2014

(51) Int. Cl. A61M 5/50; A61M 5/142

(52) U.S. Cl. CPC A61M 5/5086; A61M 5/142

(57) ABSTRACT

An infusion pump has an optical detection system for determining whether or not a cassette of an administration tubing set is properly loaded in the pump. Operation of the pump may be enabled or disabled based on a determination of the optical detection system. The optical cassette detection system includes a light emitter and a corresponding light detector on non-coincident emission and detection axes, and at least one optical element carried by the cassette. When the cassette is properly loaded in the pump, light from the emitter is redirected by the optical element from the emission axis to the detection axis for receipt by the detector, which registers an increased signal level. The detector signal is evaluated by signal evaluation electronics to determine if the detector signal level is above a predetermined threshold, indicating presence of the cassette.

### FIELD OF THE INVENTION

The present invention relates generally to infusion pumps for controlled delivery of liquid food and medications to patients. More specifically, the present invention relates to a sensor system in an infusion pump for detecting the presence or absence of a cassette by which an administration tubing set is operatively connected to the pump.

### BACKGROUND OF THE INVENTION

Programmable infusion pumps are used to carry out controlled delivery of liquid food for enteral feeding and medications for various purposes, for example pain management. In a common arrangement, an infusion pump receives a disposable administration set comprising a cassette removably received by the pump and flexible tubing connected to the cassette for providing a fluid delivery path through the pump.

The cassette itself may be intended for use with a particular infusion pump model or models, and/or with tubing having predetermined properties. In this regard, the cassette may include safety features that are designed and manufactured according to specifications determined at least in part by the intended infusion pump model and/or administration set tubing. The safety features of the cassette may cooperate with corresponding features on the matching pump, and may be manufactured according to size tolerances related to tubing diameter and flexibility. For example, the cassette may have an anti-free flow mechanism for protecting the patient from uncontrolled fluid delivery. The anti-free flow mechanism may take the form of an external pinch clip occluder actuated when the cassette is properly loaded in the pump and a door of the pump is closed. Alternatively, the anti-free flow mechanism may take the form of an internal “in-line occluder” that resides within the flow passage of the tubing, wherein a flow passage is only opened when the cassette is properly loaded in the pump and the pump door is closed.

The cassette may provide additional safety features beyond free flow protection. For example, the cassette may be matched to the pump to maintain a desired volumetric accuracy of the pump, and to ensure correct function of occlusion and air-in-line sensors used to trigger safety alarms.

In view of the safety importance of the cassette, it is desirable to provide means to detect whether or not a matching cassette is properly loaded in the pump as a precondition to enabling pump operation.

### SUMMARY OF THE INVENTION

In accordance with the present invention, an infusion pump in which an administration set is removably received is provided with an optical detection system for determining whether or not a cassette of the administration set is properly loaded in the pump. In an embodiment of the present invention, operation of the pump is disabled if a cassette is not properly loaded in the pump.

The optical cassette detection system comprises an optical emitter mounted to the pump and arranged to emit a light beam directed along an emission optical axis, and a photosensitive detector mounted to the pump so as to define a light detection optical axis different from the emission optical axis. The cassette detection system further comprises at least one optical element carried by the cassette, including an optical element positioned to receive the light beam when the cassette is properly loaded in the pump. The at least one optical element redirects at least a portion of the light beam along the detection optical axis for receipt by the photosensitive detector. The photosensitive detector generates a detector signal representing an intensity of light received thereby.

The detector signal is evaluated by signal evaluation electronics to determine if the detector signal level is above a predetermined threshold, indicating presence of the cassette. The signal evaluation electronics may be in communication with a pump controller, wherein the pump controller is programmed to disable pump operation unless a cassette is present as determined by the optical cassette detection system.

In one embodiment, the emission optical axis is parallel to the detection optical axis, and the at least one optical element includes a parallel surface beam displacer arranged to displace the light beam from the emission optical axis to the detection optical axis when the cassette is properly loaded.

In another embodiment, the at least one optical element includes a prism or a wedge causing spectral dispersion of the light beam. The photosensitive detector may be arranged and configured to detect a portion of the dispersed beam in a predetermined narrower wavelength band.

In a further embodiment, the at least one optical element includes a Porro prism that reverses the direction of the light beam.

### BRIEF DESCRIPTION OF THE DRAWINGS

The nature and mode of operation of the present invention will now be more fully described in the following detailed description of the invention taken with the accompanying drawing figures, in which:

FIG. 1 is perspective view of an infusion pump and cassette embodying a cassette detection system in accordance with an embodiment of the present invention;

FIG. 2 is a perspective view of the cassette shown in FIG. 1;

FIG. 3A is a schematic sectional view illustrating a cassette detection system formed in accordance with an embodiment of the present invention, wherein a tab of the cassette is shown prior to insertion into a tab-receiving slot of the pump;

FIG. 3B is an enlarged view corresponding to FIG. 3A, however the cassette tab is shown inserted into the pump slot;

FIG. 4 is an enlarged schematic sectional view illustrating a cassette detection system formed in accordance with an another embodiment of the present invention;

FIG. 5 is an enlarged schematic sectional view illustrating a cassette detection system formed in accordance with a further embodiment of the present invention; and

FIG. 6 is a flow diagram showing decision logic executed by the cassette detection system in accordance with an embodiment of the present invention.

### DETAILED DESCRIPTION OF THE INVENTION

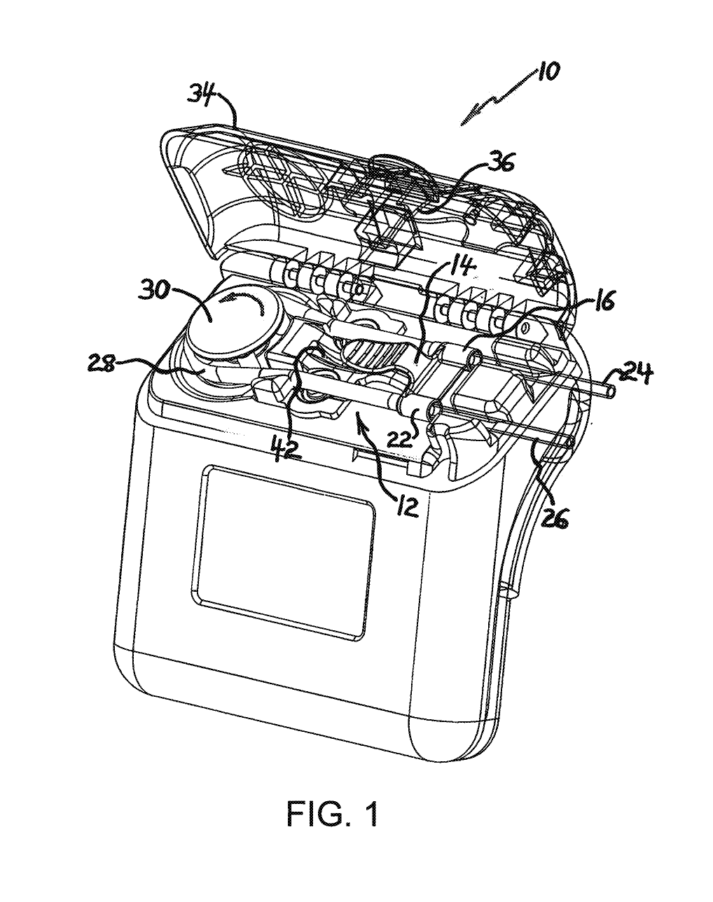

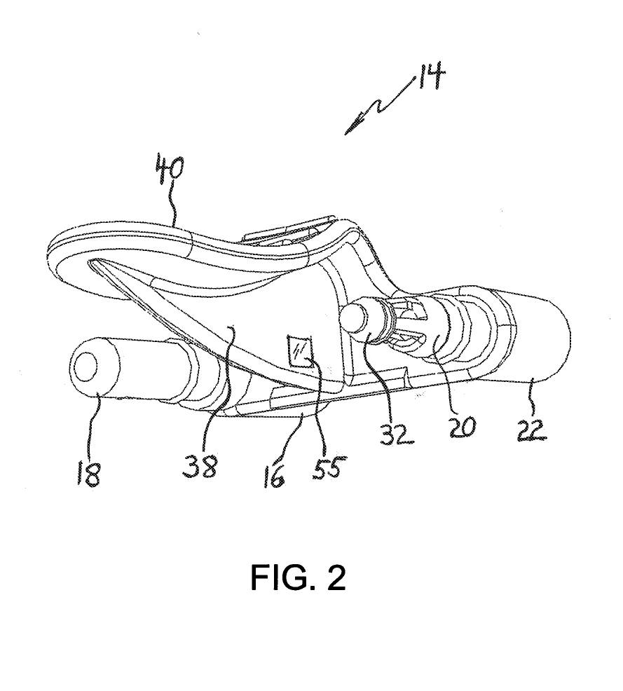

FIG. 1 shows an infusion pump 10 in which an administration set 12 is removably received. Administration set 12 includes a cassette 14, which is shown by itself in FIG. 2. Cassette 14 may include an input connector 16, an upstream loop connector 18 in flow communication with input connector 16, a downstream loop connector 20, and an output connector 22 in flow communication with downstream loop connector 20. Administration set 12 may further include inflow tubing 24 having one end mated to input connector 16 and an opposite end (not shown) connected to a fluid source, and outflow tubing 26 having one end connected to output connector 22 and an opposite end (not shown) connected to a patient. Finally, administration set 14 may further include a pumping segment of tubing 28 having one end mated to upstream loop connector 18 and an opposite end mated to downstream loop connector 20.

In the illustrated embodiment, pump 10 is a rotary peristaltic pump having a rotor 30, wherein pumping segment 28 is wrapped around rotor 30 and is engaged by angularly spaced rollers on rotor 30 as the rotor rotates to provide peristaltic pumping action forcing liquid through the tubing of administration set 12. As may be understood by reference to FIG. 1, when rotor 30 rotates in a counter-clockwise direction, liquid is moved from inflow tubing 24 through input connector 16 and upstream loop connector 18 to pumping segment 28, and then from pumping segment 28 through downstream loop connector 20 and output connector 22 to outflow tubing 26. Although the present invention is described in the context of a rotary peristaltic pump, the invention is not limited to this type of infusion pump. The invention may be practiced with any type of infusion pump that receives an administration set having a cassette.

Cassette 14 may include an in-line occluder 32 which may be incorporated into downstream loop connector 20. In-line occluder 32 prevents flow when pump door 34 is open. An actuator 36 on an underside of pump door 34 engages pumping segment 28 in a manner which opens a flow path around occluder 32 when door 34 is closed.

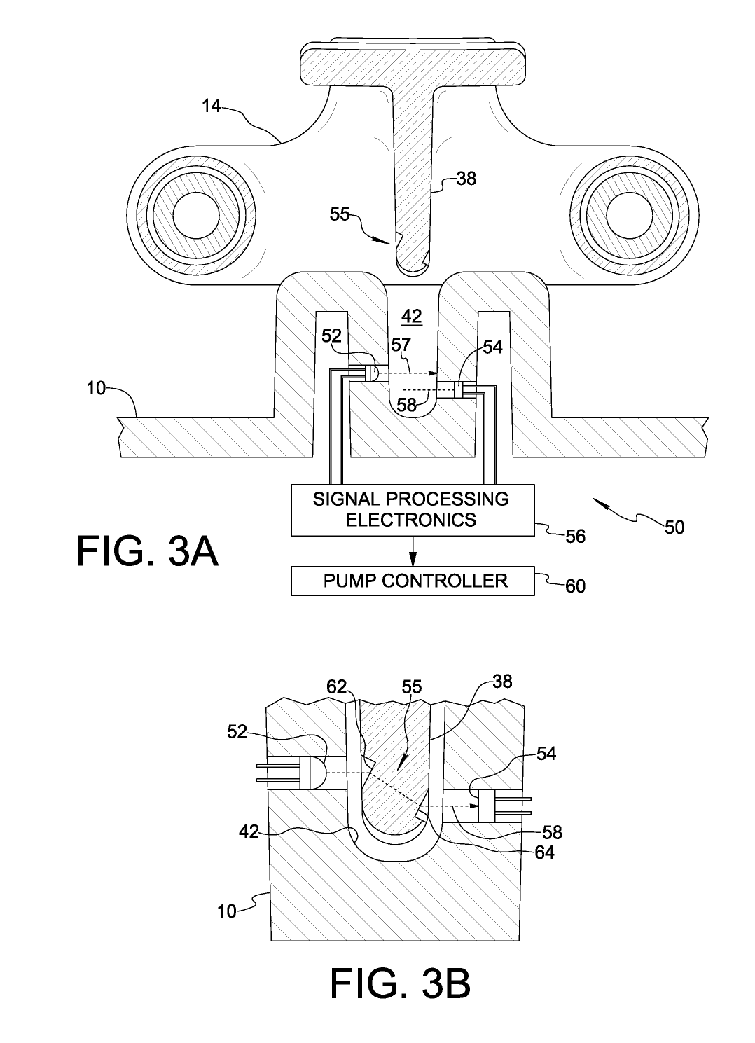

Reference is now made to FIGS. 3A and 3B. Cassette 14 includes a tab 38 depending downwardly from a ribbed thumb portion 40 of the cassette. In the present embodiment, tab 38 is a planar tab that is sized for receipt within a corresponding slot 42 in pump 10. Slot 42 may be provided at a location on pump 10 between the upstream and downstream portions of pumping segment 28, and tab 38 may be provided on an underside of thumb portion 40. For example, slot 42 may be midway between the upstream and downstream portions of pumping segment 28 and may be elongated in a direction aligned with the rotation axis of rotor 30, and tab 38 may be midway between one side of cassette 14 having input connector 16 and upstream loop connector 18 and the other side of cassette 14 having downstream loop connector 20 and output connector 22. In this symmetrical arrangement, cassette 14 is easily centered in pump 10 relative to rotor 30 during installation of administration set 12. In an embodiment of the invention, the width of slot 42 is 2.6 mm and the width of tab 38 is 1.7 mm.

Pump 10 includes an optical cassette detection system 50 operable to detect whether or not cassette 14 is properly loaded in pump 10 with cassette tab 38 present in slot 42. Cassette detection system 50 includes an optical emitter 52 and a photosensitive detector 54 each mounted in pump 10. Cassette detection system 50 further includes at least one optical element 55 carried by cassette 14. In accordance with the present invention, the at least one optical element 55 establishes an optical path from emitter 52 to photosensitive detector 54 when cassette 14 is properly loaded in pump 10. Cassette detection system 50 may also include signal processing electronics 56 connected to photosensitive detector 54 for receiving an electronic signal generated by detector 54 and evaluating the signal. Signal processing electronics 56 may be in communication with a pump controller 60, whereby operation of pump 10 may be controlled based on an evaluation of the detector signal.

In the embodiments described herein, emitter 52 and photosensitive detector 54 are each mounted in pump 10 adjacent to slot 42, and the at least one optical element 55 is part of tab 38, however other configurations and arrangements are possible. In the embodiments described herein, the at least one optical element 55 is a single optical element, however more than one optical element may be carried by cassette 14 and configured to selectively establish an optical path from emitter 52 to detector 54.

In the embodiment shown in FIGS. 3A and 3B, detector 54 is not aligned with emitter 52. Emitter 52 emits a light beam that travels along an emission optical axis 57. Detector 54 defines a light detection optical axis 58 normal to a sensing surface of the detector. Detector 54 is arranged such that detection optical axis 58 differs from emission optical axis 57. More specifically, in FIGS. 3A and 3B, emission optical axis 57 is offset from and parallel to detection optical axis 58. When cassette 14 is not properly loaded in pump 10, as shown in FIG. 3A, the light beam from emitter 52 travels into slot 42 and is absorbed and/or diffusely reflected by the slot wall. Thus, without proper loading of cassette 14, the emitted light beam is not redirected along detection optical axis 58 for receipt by detector 54. However, when cassette 14 is properly loaded in pump 10 as shown in FIG. 3B, optical element 55 is positioned in emission optical axis 57 to receive the emitted light beam. Optical element 55 redirects at least a portion of the light beam along detection optical axis 58 for receipt by detector 54.

As best seen in FIG. 3B, optical element 55 may be embodied as a beam displacing element in the nature of a plane parallel plate element having a light entry surface 62 and a light exit surface 64 parallel to light entry surface 62. Optical element 55 may be integrally formed with tab 38 or with cassette 14 as a whole. For example, cassette 14 may be molded from transparent or translucent optical grade plastic having a predetermined refractive index, wherein surfaces 62 and 64 are formed in tab 38 as external surface features. When cassette 14 is properly loaded as depicted in FIG. 3B, entry surface 62 is positioned at an oblique angle relative to emission optical axis 57 and exit surface 64 is positioned at an oblique angle relative to detection optical axis 58. Consequently, the beam from emitter 52 is redirected by refraction at the air/plastic interface provided by entry surface 62, and is again redirected by refraction at the plastic/air interface provided by exit surface 64, whereby the beam is displaced by an amount corresponding to the distance between emission optical axis 57 and detection optical axis 58.

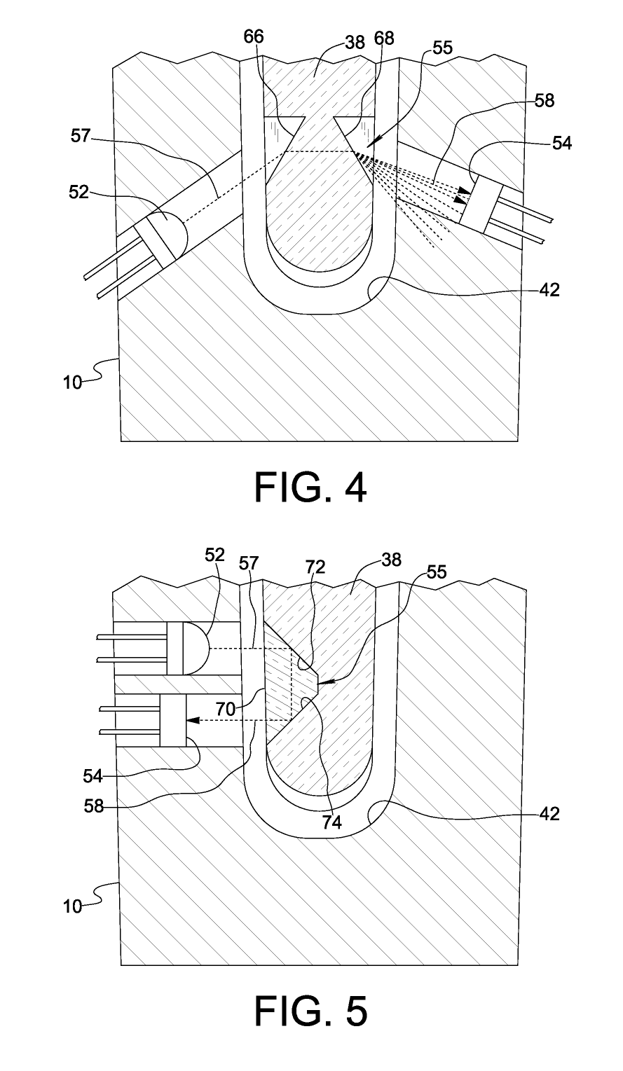

FIG. 4 shows another embodiment of the optical cassette detection system, wherein optical element 55 is in the form of a prism element having a light entry surface 66 and a light exit surface 68 nonparallel to entry surface 66. In the embodiment of FIG. 4, emitter 52 and detector 54 may be arranged such that emission optical axis 57 and detection optical axis 58 are nonparallel. For example, emission optical axis 57 and detection optical axis 58 may converge in the upward direction of FIG. 4. Light from emitter 52 may be in a relatively wide wavelength band such that it undergoes spectral dispersion as it passes through entry surface 66 and through exit surface 68, and detector 54 may be arranged and configured to detect a portion of the dispersed beam in a predetermined narrower wavelength band along detection optical axis 58. Those skilled in the art will understand that an optical wedge element may be substituted for the depicted prism element, wherein only one of the light entry surface and light exit surface is oblique to its respective optical axis.

An optical cassette detection system according to a further embodiment is illustrated in FIG. 5. In the embodiment shown in FIG. 5, the optical element 55 is in the form of a Porro prism having a light entry and exit surface 70, a first reflection surface 72, and a second reflection surface 74. Emitter 52 and detector 54 may be arranged on one side of slot 42 such that emission optical axis 57 and detection optical axis 58 extend parallel to one another and normal to light entry and exit surface 70. As will be understood from FIG. 5, the beam from emitter 52 travels along emission optical axis 57, enters the prism through surface 70, is internally reflected at a 90° angle by first reflection surface 72, is internally reflected at another 90° angle by second reflection surface 74, and exits the Porro prism through surface 70 along detection optical axis 58 for receipt by detector 54. The Porro prism may be embedded in tab 38 as shown in FIG. 5, or surfaces 70, 72, and 74 may be formed as surface features of tab 38 in a manner similar to the prior embodiments.

In the embodiments described above, a single optical element is used to redirect the light beam. However, a combination of optical elements may be used for redirection of the light beam without straying from the invention.

With respect to each embodiment, detector 54 generates a signal, for example a current or voltage signal, having a level corresponding to the intensity of light received thereby. In the unblocked condition shown in FIG. 3A, detector 54 does not receive significant light from emitter 52 and thus the detector signal level is below a predetermined threshold. When cassette 14 is properly loaded in pump 10, as shown in FIGS. 3B, 4 and 5, tab 38 occupies slot 42 and the at least one optical element 55 is positioned to redirect at least a portion of the beam from emitter 52 to reach detector 54 along optical axis 58. Consequently, when cassette 14 is loaded in pump 10, the level of the signal generated by detector 54 increases above the predetermined threshold.

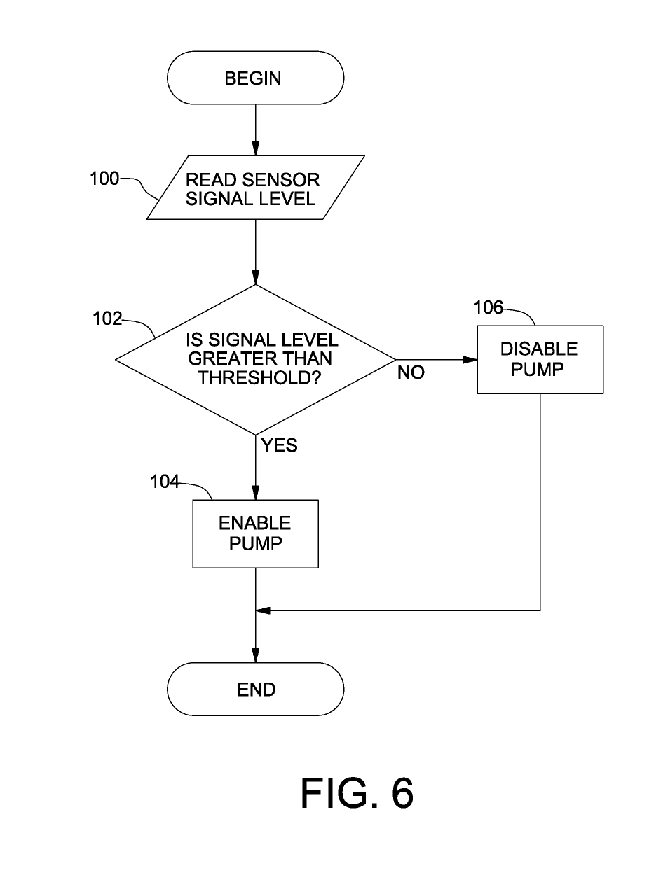

Signal processing electronics 56 evaluates the signal from detector 54 to determine if cassette 14 is properly loaded in pump 10. The signal processing and evaluation may be completely analog, or the detector signal level may be converted to a digital value and compared to a threshold in a digital comparator circuit. As illustrated in FIG. 6, operation of pump 10 may be enabled or disabled based on the determination made by signal processing electronics 56. In block 100, the level of the detector signal is read. In block 102, the signal level is compared to a predetermined threshold as the basis for a decision. If the signal level is above the threshold, presence of cassette 14 is indicated and flow branches to block 104, wherein pump operation is enabled by pump controller 60. However, if the signal level is below the threshold, flow branches to block 106 and pump operation is disabled by pump controller 60.

Emitter 52 may be a light-emitting diode (LED) or other light source, and photosensitive detector 54 may be a photodiode or other photosensitive element capable of generating an electrical signal in response to incident light. Emitter 52 and detector 54 may be chosen to operate within predetermined wavelength bands. For example, where optical element 55 is a dispersing prism, emitter 52 may be chosen to emit light in a relatively wide wavelength band, and detector 54 may have a spectral responsivity confined to a relatively narrow wavelength band or detector 54 may include a wavelength filter for selecting a relatively narrow wavelength band. Alternatively, emitter 52 may be a narrow band emitter, for example a laser diode. Likewise, detector 54 may have a spectral responsivity across a relatively wide wavelength band that includes the emission wavelength band. Emitter and detector may be optically coupled by light outside the visible spectrum, e.g. infrared or ultraviolet light. While not shown, emitter 52 and detector 54 may have lenses, fiber optics, or other optical elements associated therewith for collimating, focusing, and/or directing the beam.

Tab 38 on cassette 14 provides structure that may be used for carrying the at least one optical element 55 and positioning the at least one optical element 55 in optical cassette detection system 50. A wide variety of tab arrangements and optical detection system configurations are of course possible. The centered arrangement of a thin tab 38 on the underside of cassette 14, and the use of a thin slot 42 in pump 10, takes advantage of the tab and slot as a means for guiding and centering the cassette 14 during installation. Moreover, the cassette detection system 50 is hidden within the pump and is inconspicuous to users. Emitter 52 and detector 54 may be recessed slightly from the surface of slot 42 behind respective transparent barriers (not shown) to keep dirt and fluid away from the emitter and detector.

While the invention has been described in connection with exemplary embodiments, the detailed description is not intended to limit the scope of the invention to the particular forms set forth. The invention is intended to cover such alternatives, modifications and equivalents of the described embodiment as may be included within the spirit and scope of the invention.

What is claimed is:

1. A system for detecting loading of a cassette in an infusion pump, the system comprising:
an optical emitter mounted to the pump, the optical emitter being arranged to emit a light beam directed along an emission optical axis;
a photosensitive detector mounted to the pump, the photosensitive detector defining a light detection optical axis different from the emission optical axis, wherein the photosensitive detector generates a detector signal representing an intensity of light received thereby; and
at least one optical element carried by the cassette, the at least one optical element including an optical element positioned to receive the light beam when the cassette is properly loaded in the pump, wherein the at least one optical element redirects at least a portion of the light beam along the detection optical axis for receipt by the photosensitive detector.

2. The system according to claim 1, further comprising signal processing electronics for evaluating the detector signal to determine whether the cassette is properly loaded in the pump.

3. The system according to claim 1, wherein the at least one optical element is a single optical element.

4. The system according to claim 1, wherein the at least one optical element includes a parallel surface beam displacer.

5. The system according to claim 1, wherein the at least one optical element includes a prism or a wedge.

6. The system according to claim 5, wherein the at least one optical element includes a Porro prism.

7. The system according to claim 1, wherein the emission optical axis is parallel to the detection optical axis.

8. The system according to claim 1, wherein the light beam has a wide wavelength band, and the at least one optical element includes a dispersing prism for dispersing the light beam into a plurality of narrower wavelength bands.

9. The system according to claim 8, wherein the photosensitive detector is a narrow wavelength band detector configured for detecting one of the plurality of narrower wavelength bands.

10. The system according to claim 1, wherein the cassette includes a tab in which the at least one optical element is located, and the pump includes a slot configured to receive the tab when the cassette is properly loaded in the pump.

11. The system according to claim 10, wherein the optical emitter and the photosensitive detector are on opposite sides of the slot.

12. The system according to claim 10, wherein the optical emitter and the optical detector are on one side of the slot.

13. An infusion pump comprising:
an optical emitter arranged to emit a light beam directed along an emission optical axis; and
a photosensitive detector defining a light detection optical axis different from the emission optical axis, wherein the photosensitive detector generates a detector signal representing an intensity of light received thereby;
wherein the optical emitter is placed into optical communication with the photosensitive detector by properly loading a cassette in the infusion pump.

14. The infusion pump according to claim 13, further comprising signal processing electronics for evaluating the detector signal to determine whether the cassette is properly loaded in the pump.

15. A cassette to be loaded in an infusion pump for operatively connecting an administration set to the pump, the cassette comprising:
an input tubing connector;
an output tubing connector;
at least one optical element spaced from the input tubing connector and the output tubing connector, wherein the at least one optical element is configured to redirect light from a first optical axis to a second optical axis different from the first optical axis.

16. The cassette according to claim 15, wherein the cassette is a one-piece molded part, and the at least one optical element includes an optical element integrally formed with the cassette.

17. The cassette according to claim 16, wherein the cassette is molded from transparent or translucent plastic.

18. The cassette according to claim 15, wherein the at least one optical element includes an optical element embedded in the cassette.
